(19) United States
(12) Patent Application Publication
(10) Pub. No.: US 2025/0057578 A1
(43) Pub. Date: Feb. 20, 2025

(54) SYSTEMS AND METHODS FOR LOCALIZED ABLATION WITH A CIRCULATING FLUID CATHETER

(71) Applicant: INTUITIVE SURGICAL OPERATIONS, INC., Sunnyvale, CA (US)

(72) Inventors: Erik M. Henne, Mercer Island, WA (US); Ryan R. Bacsik, Seattle, WA (US); Robert L. Barry, Kirkland, WA (US); Jacqueline L. Corso, Kirkland, WA (US); Joshua P. Kroon, Seattle, WA (US); Cassandra R. Saleira, Bothell, WA (US); Jesse L. Via, Seattle, WA (US); Alexander M. Vanden Bussche, Seattle, WA (US)

(21) Appl. No.: 18/728,553

(22) PCT Filed: Jan. 11, 2023
(86) PCT No.: PCT/US2023/060446

§ 371 Date: Jul. 12, 2024

Related U.S. Application Data
(60) Provisional application No. 63/299,317, filed on Jan. 13, 2022.

(51) Int. Cl. A61B 18/04; A61B 18/00

(52) U.S. Cl. CPC A61B 18/04; A61B 2018/0022; A61B 2018/00577; A61B 2018/00744; A61B 2018/044

(57) ABSTRACT

A system comprises an elongate instrument including a first and second channels. The system also comprises an expandable device coupled to the elongate instrument. The expandable device is in fluid communication with the first and second channels. The system also comprises a pump for circulating a fluid through the first and second channels and a heating system for heating the fluid. The system also comprises a drainage system for removing the fluid from the expandable device and restricting drainage flow. A control system controls temperature and delivery of the heated fluid through the first channel and the second channel to heat the elongate instrument and the expandable device with the heated fluid while maintaining the expandable device in an uninflated configuration, inflate the expandable device with the heated fluid, ablate an anatomic passage with the inflated and heated expandable device, and deflate the expandable device.

### CROSS-REFERENCED APPLICATIONS

This application claims priority to and benefit of U.S. Provisional Application No. 63/299,317, filed Jan. 13, 2022 and entitled “Systems and Methods for Localized Ablation With a Circulating Fluid Catheter,” which is incorporated by reference herein in its entirety.

### FIELD

Examples described herein relate to systems and methods for endoluminal thermal treatment. More particularly, examples may relate to localized ablation using a circulating fluid catheter.

### BACKGROUND

Minimally invasive medical techniques may generally be intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions an operator may insert minimally invasive medical instruments such as therapeutic instruments, diagnostic instruments, imaging instruments, and surgical instruments. Some minimally invasive medical instruments may be used to perform ablation. Improved ablation instruments are needed to control the duration, location, and/or uniformity of heat delivery.

### SUMMARY

The following presents a simplified summary of various examples described herein and is not intended to identify key or critical elements or to delineate the scope of the claims.

In some examples, system comprises an elongate instrument including a first and second channels. The system also comprises an expandable device coupled to a distal portion of the elongate instrument. The expandable device is in fluid communication with the first and second channels. The system also comprises a pump system for circulating a fluid through the first and second channels and a heating system for heating the fluid. The system also comprises a control system coupled to the heating system and the pump system. The control system is configured to control temperature and delivery of the heated fluid through the first channel and the second channel to heat the elongate instrument and the expandable device with the heated fluid while maintaining the expandable device in an uninflated configuration, inflate the expandable device with the heated fluid, ablate an anatomic passage with the inflated and heated expandable device, and deflate the expandable device.

In some examples, a method is disclosed for controlling flow of a fluid through a first channel and a second channel of an elongated instrument to ablate a target tissue with an expandable device coupled to the elongate instrument. The method comprises heating the fluid and heating the elongate instrument and the expandable device by circulating the heated fluid through the first channel and second channel while maintaining the expandable device in an uninflated configuration. The method also includes inflating the expandable device by circulating the heated fluid through the first channel and the second channel and ablating the target tissue by contacting a wall of an anatomic passageway with the inflated and heated expandable device. The method also includes deflating the expandable device by changing the flow of the heated fluid through the first channel and the second channel.

It is to be understood that both the foregoing general description and the following detailed description are illustrative and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1 is a simplified diagram of a patient anatomy, according to some examples.

FIG. 2A is a schematic illustration of a medical system, according to some examples.

FIG. 2B is a schematic illustration of a medical system, according to some examples.

FIG. 3A illustrates a catheter with an expandable device in an uninflated state, according to some examples.

FIG. 3B illustrates the catheter of FIG. 3A with the expandable device in a partially inflated state, according to some examples.

FIG. 3C illustrates the catheter of FIG. 3A with the expandable device in an inflated state, according to some examples.

FIG. 3D illustrates the catheter of FIG. 3A with the expandable device in a partially deflated state, according to some examples.

FIG. 3E illustrates the catheter of FIG. 3A with the expandable device in a deflated state, according to some examples.

FIG. 4 is a flowchart illustrating a method of endoluminal thermal treatment using a catheter and expandable device, according to some examples.

FIG. 5A illustrates a perspective view of a catheter with an outlet and an inlet for conveying heated fluid, according to some examples.

FIG. 5B illustrates a perspective view of a catheter with an outlet and an inlet for conveying heated fluid, according to some examples.

FIG. 5C illustrates a perspective view of a catheter and an adjustable outer sheath, according to some examples.

FIG. 5D illustrates a perspective view of a catheter with an outlet and an inlet for conveying heated fluid and with cuffs to secure the expandable device, according to some examples.

FIG. 5E is a cross-sectional view of the coupling between the expandable device and the cuffs.

FIGS. 6A-6C illustrate cross-sectional views of catheters having various channel configurations, according to some examples.

FIG. 7 is a graph charting inflation volume vs. time throughout endoluminal thermal treatment via a catheter and expandable device, according to some examples.

FIG. 8 is a robot-assisted medical system, according to some examples.

Examples of the present disclosure and their advantages are best understood by referring to the detailed description that follows. It should be appreciated that like reference numerals are used to identify like elements illustrated in one or more of the figures, wherein showings therein are for purposes of illustrating examples of the present disclosure and not for purposes of limiting the same.

### DETAILED DESCRIPTION

The technology described herein provides techniques and treatment systems for endoluminal thermal treatment of diseased tissue. Although the examples provided herein may refer to treatment of lung tissue and pulmonary disease, it is understood that the described technology may be used in treating artificially created lumens or any endoluminal passageway or cavity, including in a patient trachea, colon, intestines, stomach, liver, kidneys and kidney calices, brain, heart, circulatory system including vasculature, fistulas, and/or the like. In some examples, treatment described herein may be referred to as endobronchial thermal fluid treatment and may be used in procedures to treat lung tumors and/or chronic obstructive pulmonary disease (COPD) that may include one or more of a plurality of disease conditions including chronic bronchitis, emphysema, and bronchiectasis.

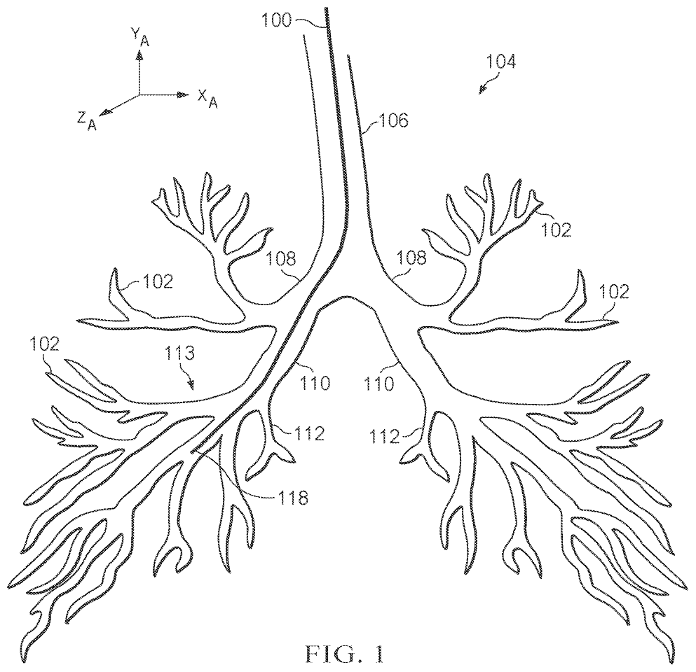

FIG. 1 illustrates an elongated medical instrument system 100 extending within branched anatomic passageways or airways 102 of an anatomical structure 104. In some examples the anatomic structure 104 may be a lung and the passageways 102 may include the trachea 106, primary bronchi 108, secondary bronchi 110, and tertiary bronchi 112. The anatomic structure 104 has an anatomical frame of reference (X_{A}, Y_{A}, Z_{A}). A distal end portion 118 of the medical instrument 100 may be advanced into an anatomic opening (e.g., a patient mouth) and through the anatomic passageways 102 to perform a medical procedure, such as an endoluminal thermal energy treatment, at or near target tissue located in a region 113 of the anatomic structure 104. The elongated medical instrument system 100 may include an ablation system for ablating diseased tissue. The ablation system may include a heated device that is expandable to initiate contact with a wall of an anatomic passageway 102. During ablation, cellular and structural changes in the epithelium and sub-epithelium may be induced. The ablation may cause tissue reduction, including destruction of goblet cells and cilia in lung tissue. In some examples, the cellular matrix may be preserved to allow for later regrowth of healthy cells. In some examples, the tissue reaction may occur entirely during the application of the heat, and in other examples, the tissue damage may develop over a period of time as the anatomy responds to the injury caused by the heat. In the lung, an ablation may be used to treat a variety of pulmonary conditions including lung tumors, chronic obstructive pulmonary disease, chronic bronchitis, emphysema, and bronchiectasis.

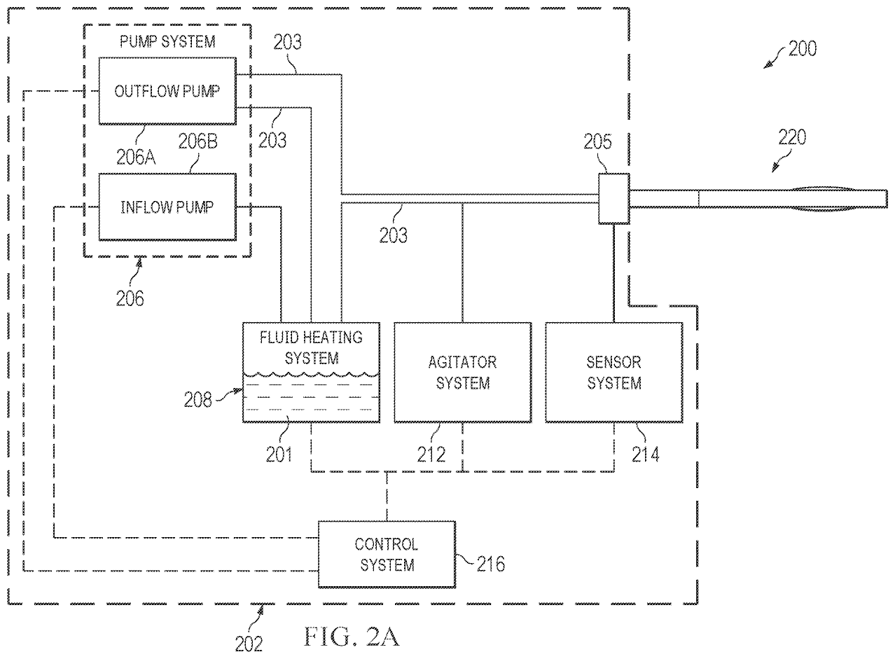

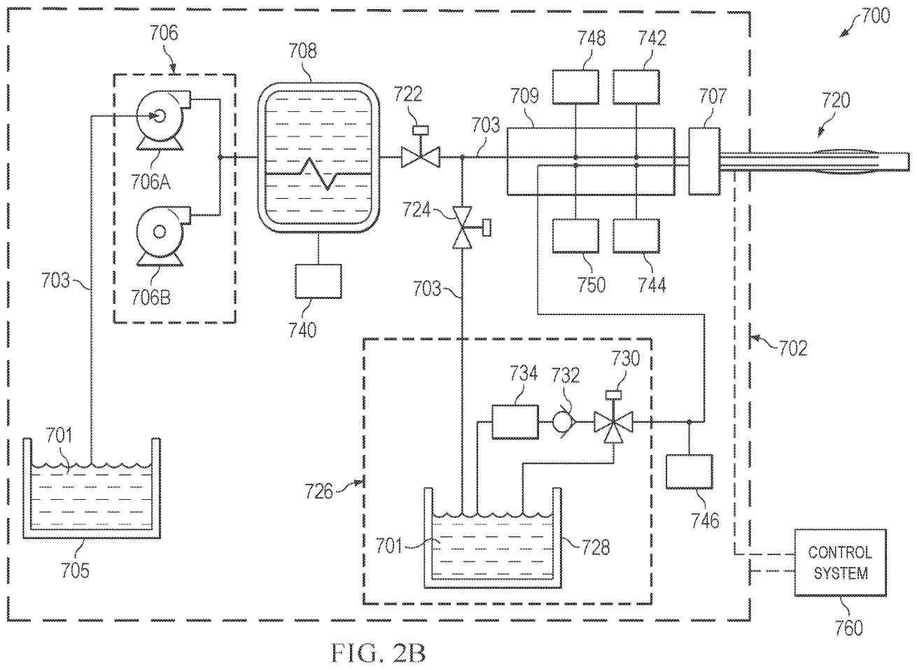

FIG. 2A illustrates an ablation system 200 including a fluid system 202 coupled to or otherwise in fluid communication with a treatment device 220. The treatment device 220 may be, for example, a catheter which may be an example of the elongated medical instrument system 100 of FIG. 1. In this example, when a distal portion of the treatment device 220 is positioned within an anatomic passageway 102, a fluid 201 may circulate through the ablation system 200. The fluid 201 may include any of a variety of different fluid substances including water (e.g., distilled water), saline, or a gas such as air. In some examples, the fluid 201 may include a radiopaque substance. The substances listed above are not limiting, but examples. Components of the fluid system 202 may be coupled to each other and/or to the treatment device 220 by conduit 203 or other fluid passageways. The fluid system 202 may include a pump system 206 having an outflow pump 206A and an inflow pump 206B. The pump system is not limited to two pumps, and a plurality of pumps may be used. Additionally, or alternatively, the pump system may include a bi-directional pump. In some examples, the outflow and/or inflow pumps may include a syringe pump.

The fluid system 202 may also include a fluid heating system 208 that may be in fluid communication with the outflow pump 206A and inflow pump 206B through the fluid conduit 203. Fluid heating system 208 may include a fluid reservoir, heating element or generator, power source, insulation, temperature sensor, temperature regulation systems and/or other components to contain, heat, and/or control the temperature of the fluid 201. The fluid heating system 208 may increase, decrease, or maintain the temperature of the fluid 201 to predetermined, operator selected, or otherwise suitable target temperature for delivering to the treatment device 220. In some examples, the fluid heating system 208 may heat the fluid in a reservoir with resistive heating, radiofrequency heating, ultrasonic heating, laser heating, magnetic heating, and/or microwave heating.

The fluid system 202 may also include an agitator system 212 that may agitate the fluid 201 that is delivered to the treatment device 220 to reduce temperature variations in the delivered fluid. The agitator system 212 may include an oscillating or vibrating device that creates a waveform or duty cycle in the fluid flow or rapidly agitates the fluid. In some examples, the oscillating device may be a solenoid with a preset or operator controllable duty cycle. In some examples, the agitator system 212 may include a syringe that is coupled to a fluid conduit 203 with an adapter. The syringe may be connected to an oscillating solenoid on a duty cycle to create a wave form in the flow rate. The wave form may induce chaotic mixing of the fluid within expandable device 300. In some examples, the agitator system may be coupled between fluid heating system 208 and treatment device 220. In other examples, the components of the agitator system may be located in a fluid reservoir of the fluid heating system 208.

The fluid system 202 may also include a sensor system 214 in electrical communication with treatment device 220, pump system 206, fluid heating system 208, and/or agitator system 212. The sensor system 214 may comprise, but is not limited to a temperature sensor, a pressure sensor, a volumetric flow sensor, a capacitive sensor, or an imaging system. The temperature sensor may be configured to sense temperature of the fluid 201 flowing into and/or out of the treatment device 220. The pressure sensor may be configured to sense a pressure of the fluid 201 flowing into and/or out of the treatment device 220. The volumetric flow sensor may sense the flow rate of the fluid 201 flowing into and/or out of the treatment device 220. The flow rate may be used to determine a volume of fluid 201 flowing into and/or out of the treatment device 220 over a given period. Further, a plurality of volumetric flow rates may be measured and compared. A capacitive sensor may measure the fluid capacitance at any location within system 200. In other examples the sensor system 214 may include an imaging system that provides image data that may be used to monitor, for example, obstructions, agitation, tissue contact, flow rate, temperature, pressure or other characteristics associated with the system 200 and the fluid 201 flowing therethrough. In some examples, the imaging system may be used for placement of the treatment device 220 and navigation through passageway 102. The sensor system 214 may include any combination of sensors to achieve the desired sensing. In some examples, the sensors may be located on the treatment device 220 or at any suitable location within the fluid system 202.

The fluid system 202 may be coupled to the treatment device 220 by a hub 205. In some examples, the hub 205 may comprise a single hub with two ports, one being a fluid inlet port for introducing fluid 201 into the treatment device 220 and one being an outlet port through which fluid 201 exits the treatment device 220. In some examples, the hub may include a single port or more than two ports for conveying fluid. Flow may be reversed in any of the fluid inlet or outlet ports. For example, fluid 201 may flow through a fluid inlet port or fluid outlet port in any direction. In some examples, a fluid inlet port may function as a fluid outlet port, and a fluid outlet port may function as a fluid inlet port. In some examples the fluid inlet port and fluid outlet ports may both introduce fluid 201 into treatment device 220. Similarly, the fluid inlet port and fluid outlet port may both extract fluid 201 from the treatment device 220.

In some examples, the fluid 201 leaving the treatment device 220 via the hub 205 is pumped by the outflow pump 206A through the fluid conduit 203 into the fluid heating system 208 where the fluid 201 is reheated to a target temperature. The fluid 201 may be withdrawn from the fluid heating system 208 by the inflow pump 206B, and the withdrawn fluid may optionally be agitated by the agitator system 212 before being delivered to the treatment device 220.

The fluid system 202 may also include a control system 216 in electrical communication with other components of the fluid system 202 including the pump system 206, the fluid heating system 208, the agitator system 212, and the sensor system 214. The control system 216 may use information received from the components of the fluid system 202 and/or the treatment device 220 to monitor, adjust, or other control the temperature, flow rate, pressure, agitation, or other characteristics of the fluid 201 and its flow. In some examples, the fluid system 202 may include valves, circulation pumps, user interface devices, power supplies, and electrical controls that also may be monitored and controlled by the control system 216. In some examples, dedicated valves may be used with any or all components of the fluid system. In some examples, one or more multi-way valves may be used to control the flow of any of the components of the fluid system. In some examples, the temperature, flow rate, flow initiation, flow termination, or other control aspects of the fluid system may be controlled by a control system of a robot-assisted medical system (e.g., control system 812). In some examples, separate pumps may be placed in-line with the separate fluid reservoirs to control different fluid flow rates.

The treatment device 220 may be delivered through a lumen of an elongated delivery instrument (not shown). The elongated delivery instrument may be an elongate flexible delivery instrument that is steerable via manual or robot-assisted control. For example, the delivery instrument may be a component of a bronchoscope or a navigable, robot-assisted medical instrument system. In other examples, the treatment device 220 may be a component of the bronchoscope or a navigable, robotic-assisted medical instrument system.

FIG. 2B illustrates an ablation system 700 including a fluid system 702 coupled to or otherwise in fluid communication with a treatment device 720. The treatment device 720 may be, for example, a catheter which may be an example of the elongated medical instrument system 100 of FIG. 1. In this example, when a distal portion of the treatment device 720 is positioned within an anatomic passageway 102, a fluid 701 may circulate through the ablation system 700. The fluid 701 may include any of a variety of different fluid substances including water (e.g., sterile distilled water), saline, or a gas such as air. In some examples, the fluid 701 may include a radiopaque substance. The substances listed above are not limiting, but examples. Components of the fluid system 702 may be coupled to each other and/or to the treatment device 720 by conduit 703 or other fluid passageways. The fluid system 702 may include a pump system 706 having an inflow pump 706A and a delivery pump 706B. In this example, the inflow pump 706a may include a rotary pump that generates a suction force to move the fluid 701 from a fluid reservoir 705. The rotary pump may have a piston action that creates a percussion that circulates or agitates the fluid 701. In this example the delivery pump 706B may include a syringe pump used to inflate an expandable device (e.g. the expandable device 300) of the treatment device 720. The syringe pump may deliver precise volumes of fluid to the treatment device, with a linear movement and a relatively smooth fluid flow. A syringe pump may allow for quick inflation or deflation. For example, a syringe pump may deliver short bursts of fluid at high pressures to quickly inflate an expandable device in the treatment device 720. The pump system is not limited to two pumps, and a plurality of pumps may be used.

The fluid system 702 may also include a fluid heating system 708 that may be in fluid communication with the inflow pump 706A and the delivery pump 706B through the fluid conduit 703. For example, fluid 701 may be drawn from the fluid reservoir 705 into the fluid heating system 708 by the inflow pump 706A and may be conveyed from the fluid heating system 708 to the treatment device 720 by the delivery pump 706B. Fluid heating system 708 may include a fluid reservoir, heating element or generator, power source, insulation, temperature sensor, temperature regulation systems and/or other components to contain, heat, and/or control the temperature of the fluid 701. The fluid heating system 208 may increase, decrease, or maintain the temperature of the fluid 701 to predetermined, operator selected, or otherwise suitable target temperature for delivering to the treatment device 720. In some examples, the fluid heating system 208 may heat the fluid in a reservoir with resistive heating, radiofrequency heating, ultrasonic heating, laser heating, magnetic heating, and/or microwave heating.

In this example, the fluid system 702 may include a flow control devices such as a two-way delivery valve 722 to control flow from the fluid heating system 708 to the treatment device 720 and a two-way purge valve 724 to drain fluid from conduit 703 between the fluid heating system 708 and the treatment device 720.

The fluid system 702 may also include a drainage system 726 which may include a collection reservoir 728, a flow control device 730, a flow control device 732, and a flow restriction device 734. Fluid 701 may flow from the treatment device 720 and may be diverted by the flow control device 730, which may be a three-way valve, to the collection reservoir 728 or toward a flow control device 732, which may be a check valve, and toward the flow restriction device 734. When a spike in fluid flow rate causes the expandable device of the treatment device 720 to inflate, the flow restriction device 734 may restrict flow entirely or almost entirely, providing high back pressure. Similarly, balloon deflation may occur when a drop in flow rate triggers the flow restriction device 734 to open completely or almost completely, providing no back pressure. The flow restriction device 734 may include an orifice selectable based on the size, volume, or other characteristics of the expandable device of the treatment device 720. In some examples, the orifice may be manually selected by an operator. In other examples, the orifice may be selected based on an identification of the expandable device of the treatment device 720 or a sensed property of the ablation system 700. Fluid purged by the purge valve 724 or withdrawn from the treatment device 720 may flow to the collection reservoir 728. In some examples, the drainage system 726 may dispose of the fluid collected in the collection reservoir 728. In other examples, the drainage system may include filters, chemical treatment system, sensors, pumps, fluid control devices, and/or evaluation systems for recycling the collected fluid to be reheated by the fluid heating system 208 and returned to the treatment device 720. In some examples, the fluid reservoir 705 may be connected to or part of the collection reservoir 728.

The fluid system 702 may be coupled to the treatment device 720 by a hub 709 and connector system 707. The connector system 707 may include a mechanical coupler for connecting the treatment device 720 to the fluid system 702 to maintain a secure connection. In some examples, the hub 709 may comprise a single hub with two ports, one being a fluid inlet port for introducing fluid 701 into the treatment device 720 and one being an outlet port through which fluid 701 exits the treatment device 720. In some examples, the hub may include a single port or more than two ports for conveying fluid. Flow may be reversed in any of the fluid inlet or outlet ports. For example, fluid 701 may flow through a fluid inlet port or fluid outlet port in any direction. In some examples, a fluid inlet port may function as a fluid outlet port, and a fluid outlet port may function as a fluid inlet port. In some examples the fluid inlet port and fluid outlet ports may both introduce fluid 701 into treatment device 720. Similarly, the fluid inlet port and fluid outlet port may both extract fluid 701 from the treatment device 720.

The fluid system 702 may also include a sensor system comprising sensors 740-750. The sensor 740 may be a temperature sensor that senses a temperature of the fluid 701 in the fluid heating system 708. The sensor 742 may be a temperature sensor that senses a temperature of the fluid 701 as it exits the fluid system 702 and enters the treatment device 720. The sensor 742 may be a component of the hub 709. The sensor 744 may be a temperature sensor that senses a temperature of the fluid 701 as it exits the treatment device 720 and re-enters the fluid system 702. The sensor 744 may be a component of the hub 709. The sensor 746 may be a temperature sensor that senses a temperature of the fluid 701 as it enters the drainage system 726. The sensor system may also include a pressure sensor 748 that senses the pressure of fluid 701 entering the treatment device 720 and a pressure sensor 750 that senses the pressure of fluid 701 exiting the treatment device 720. The sensors 748, 750 may be components of the hub 709. The pressure sensors 748, 750 may form a closed-loop pressure control system to control the pressure in the treatment device 720 and particularly the expandable device portion of the treatment device. The sensor system may be in electrical communication with treatment device 720, the pump system 706, fluid heating system 708, the flow control devices 722, 724, and/or the drainage system 726. The sensor system may include fewer sensors or additional sensors including additional temperature sensors, additional pressure sensors, a volumetric flow sensor, a capacitive sensor, or an imaging system.

The ablation system 700 may also include a control system 760 in electrical communication with the treatment device 720 and components of the fluid system 702 including the pump system 706, the fluid heating system 708, the drainage system 726, the flow control devices 722, 724, and the sensor system. The control system 760 may use information received from the components of the fluid system 702 and/or the treatment device 720 to monitor, adjust, or other control the temperature, flow rate, pressure, agitation, or other characteristics of the fluid 701 and its flow. In some examples, the fluid system 702 may include other valves, circulation pumps, user interface devices, power supplies, and electrical controls that also may be monitored and controlled by the control system 760. In some examples, dedicated valves may be used with any or all components of the fluid system. In some examples, the temperature, flow rate, flow initiation, flow termination, or other control aspects of the fluid system may be controlled by a control system of a robot-assisted medical system (e.g., control system 812).

The treatment device 720 may be delivered through a lumen of an elongated delivery instrument (not shown). The elongated delivery instrument may be an elongate flexible delivery instrument that is steerable via manual or robot-assisted control. For example, the delivery instrument may be a component of a bronchoscope or a navigable, robot-assisted medical instrument system. In other examples, the treatment device 720 may be a component of the bronchoscope or a navigable, robotic-assisted medical instrument system.

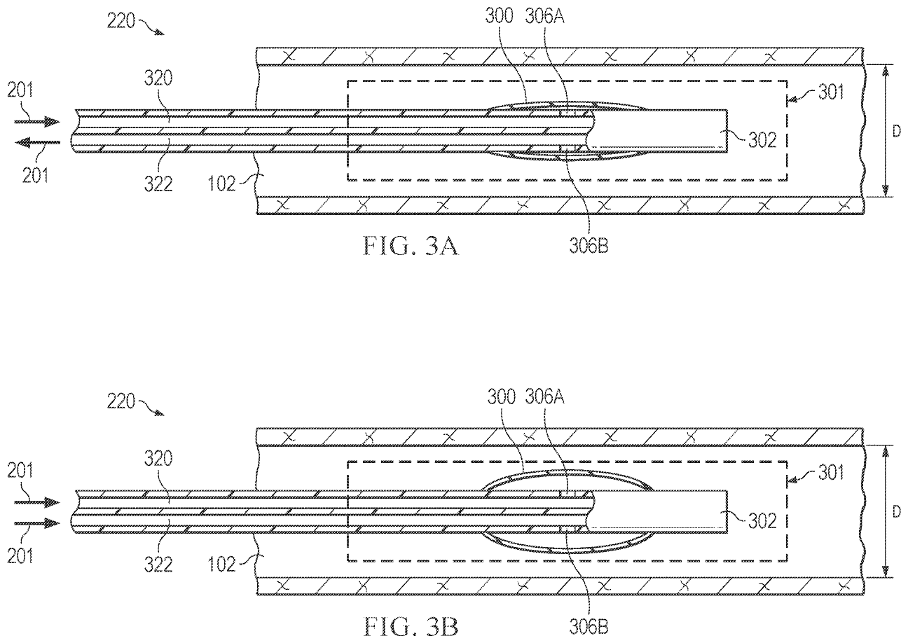

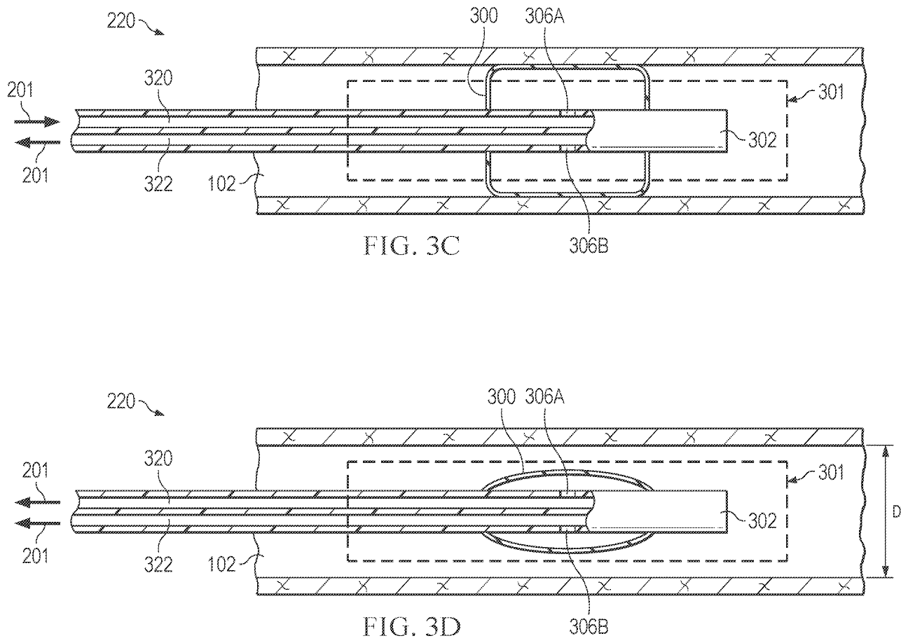

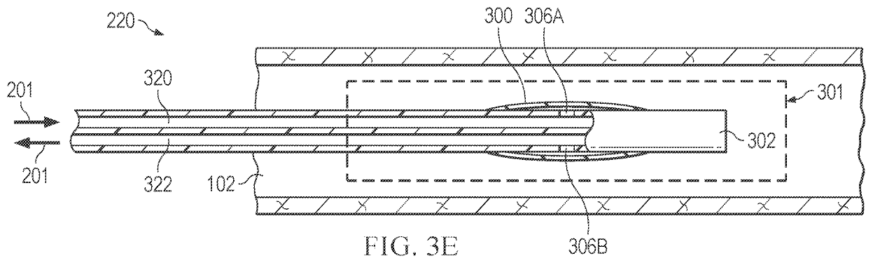

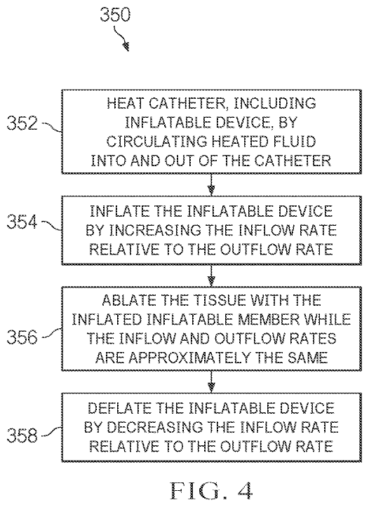

FIG. 3A illustrates components of the treatment device 220 in greater detail. The catheter system 220 includes an elongated shaft 302 with a distal portion 301 to which an expandable device 300 that may be inflated is coupled. In this example, the elongated shaft 302 may extend through the expandable device 300. The proximal end of elongated shaft 302 may be coupled to and in fluid communication with the fluid conduit 203 of fluid system 202 via the hub 205. In other examples, the treatment device 220 may be connected to the fluid system 702 or another type of fluid system. The expandable device 300 may be a compliant or a semi-compliant balloon that conforms to the shape of the passageway 102 having a diameter D. The expandable device 300 may be formed, for example, from polyisoprene, silicone, polymer, rubber, and/or other suitable materials having high elasticity and good bonding properties. The diameter D may be inconsistent near the location of the contact and treatment, and the compliance of expandable device 300 may allow for conformity based on the shape of passageway 102. In some examples, the elongated shaft 302 may be formed from a polyether block amide material (e.g., PEBAX) or another suitable thermoplastic elastomer material.

The elongated shaft 302 has a channel 320 (e.g., a first channel) to convey the fluid 201 to the expandable device 300 from a proximal portion of elongated shaft 302 coupled to the fluid system 202 or from the expandable device 300 to the proximal portion of the elongated shaft 302. The elongated shaft 302 also has a channel 322 (e.g., a second channel) to convey the fluid 201 to the expandable device 300 from a proximal portion of elongated shaft 302 coupled to the fluid system 202 or from the expandable device 300 to the proximal portion of the elongated shaft 302. The direction of fluid flow may be related to the stage of a medical procedure using the ablation system 200. The expandable device 300 may be in fluid communication with the channel 320 via a fluid port 306A and with the channel 322 via a fluid port 306B such that the fluid 201 may be introduced to the interior of the expandable device 300 through one or both of the fluid ports 306A, 306B and/or the fluid 201 may be evacuated from the expandable device 300 through one or both of the fluid ports 306A, 306B.

FIGS. 3A-3E illustrate a process for treating the anatomy including the passageway 102 with the ablation system 200. This process may be used to treat a variety of conditions including, for example, bronchitis, bronchiectasis, cancer, and/or emphysema. For some conditions, the target tissue for treatment with the ablation system 200 may be the tissue directly contacted by the expandable device 300 or in close proximity, e.g. at approximately the same location as the expandable device 300 but radially spaced within a body lumen wall. In other examples the target tissue for treatment, for example a lung tumor or diseased region of tissue, may be located distally of or downstream from the positioned distal end of the elongated shaft 302. Thus, the target tissue may be treated by the ablation even when longitudinally spaced apart from the tissue that comes into direct contact with the expandable device by ablating blood vessels providing blood to the target tissue and reducing or eliminating the blood flow to the target tissue. In additional examples, the target tissue can be treated both by ablating blood vessels downstream from the target tissue and ablating at a longitudinal location at or near the target tissue. FIG. 4 is a flowchart illustrating an ablation method 350 that may be performed with an ablation system, such as ablation system 200. As compared to examples in which an inflatable ablation member houses a heating element, heating an expandable device with the circulating heated fluid, as in ablation system 200, may provide an ablation member that is more compliant and better able to navigate tortuous passageways, is less fragile, has a reduced electrical hazard, and/or has a more controllable temperature.

The method 350 is illustrated as a set of operations or processes that may be performed in the same or in a different order than the order shown in FIG. 4. One or more of the illustrated processes may be omitted in some examples of the method. Additionally, one or more processes that are not expressly illustrated in FIG. 4 may be included before, after, in between, or as part of the illustrated processes. In some examples, one or more of the processes of method 400 may be implemented, at least in part, by a control system executing code stored on non-transitory, tangible, machine-readable media that when run by one or more processors (e.g., the processors of a control system) may cause the one or more processors to perform one or more of the processes.

Prior to initiating method 350 a distal portion of a device (e.g., portion 301 of treatment device 220) may be positioned in an anatomic passageway (e.g., a passageway 102). Pulmonary blood vessels or vasculature may extend alongside the anatomic passageway. The positioning of the catheter may be performed with a robot-assisted medical system or may be manually performed with a bronchoscope or other endoscopic instrument by a clinician.

At a process 352, a portion of a treatment device (e.g. expandable device coupled to elongated shaft) may be heated by circulating heated fluid into and out of the treatment device. This may also be considered a pre-heating stage of the method 350. The expandable device may be heated by the circulating fluid but may remain substantially uninflated. In some examples, the fluid circulation may be at approximately the same fluid inflow and outflow rates. In other examples, the outflow rate may be greater than the inflow rate. The process 352 may be a pre-heating stage of the ablation method 350, allowing the elongated shaft and the expandable device to become heated before making substantial contact with the tissue wall of passageway 102. For example and with reference to FIG. 3A, the elongated shaft 302 and expandable device 300 may be heated by conveying fluid 201 from the fluid system 202, through the channel 320, and into the expandable device 300 via the port 306A. The fluid 201 may be withdrawn from the expandable device 300 via the port 306B and conveyed through the channel 322 back to the fluid system 202 where it is reheated and prepared for recirculation. The heated fluid 201 may be conveyed through the channels 320, 322 at approximately the same inflow and outflow rates, and thus the expandable device 300 may remain substantially uninflated or minimally inflated. In one example, the fluid 201 may have an inflow rate of 30 mL/min through the channel 320 and an outflow rate of 30 mL/min through the channel 322. In some examples the heated fluid 201 may be conveyed through channel 322 with an outflow rate that is greater than an inflow rate through channel 320, and the expandable device 300 may remain uninflated. For example, the fluid may have an inflow rate of 20 mL/min through the channel 320 and an outflow rate of 40 mL/min through the channel 322. Many different inflow and outflow rates can be used to preheat the catheter and expandable device, depending, for example, on the nature of the passageway 102, desired temperature of expandable device 300, and desired length of heating process 352. In some examples the duration of heating during process 602 takes approximately 1 to 60 seconds. In some examples, heating expandable device 300 may take approximately 20 seconds. A temperature of expandable device 300 may reach between approximately 50 and 99.99 degrees Celsius during the process 352. In one example, expandable device 300 is heated to between 60 and 70 degrees Celsius during the process 352.

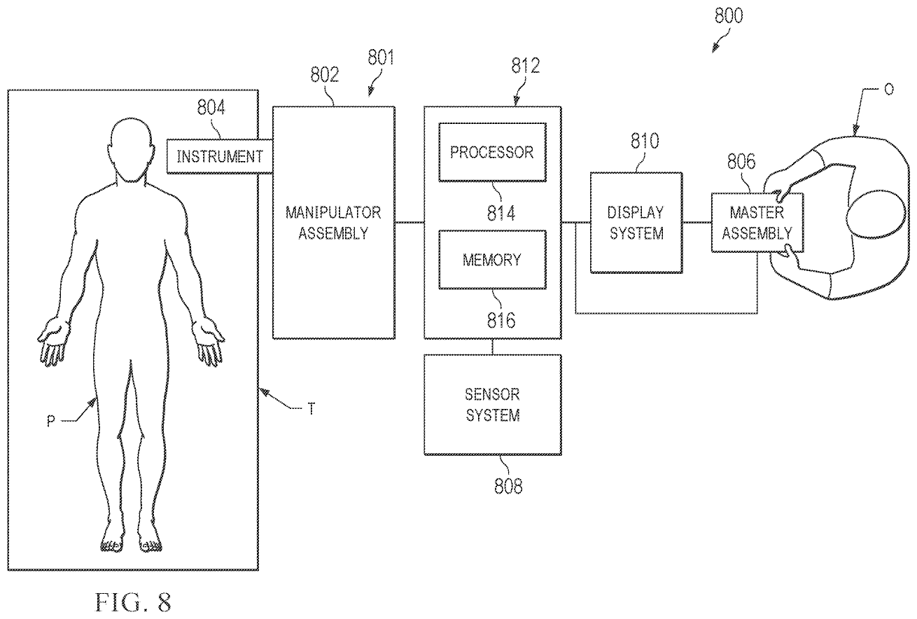

The desired temperature for expandable device 300 may vary from treatment to treatment depending on patient anatomic characteristics, disease state, or other variables. In some examples, the expandable device in an uninflated or under inflated state may be easily be navigated through the passageway 102. In some examples, the control system 216 (or control system 812 of the robot assisted medical system 800 described in FIG. 8) may move the treatment device 220 through the anatomical structure 104 while the expandable device is in an uninflated or underinflated state. In some examples, a suction force may be applied to the expandable device 300 to ensure it is uninflated while it is heated and navigating within passageways 102.

At process 354, the expandable device is inflated by increasing the inflow rate relative to the outflow rate. FIG. 3B illustrates a partially inflated expandable device 300. For example and with reference to FIG. 3B, the expandable device 300 may be inflated by increasing inflow rate of the fluid 201 through the channel 320 relative to the outflow rate of the fluid 201 through the channel 322. In other examples, channel 320 and channel 322 may both be used for inflow. For example, an inflow rate may be 200 mL/min through the channel 320 and an inflow rate may be 200 mL/min through the channel 322. In this example, providing an inflow rate through the channel 322 that is approximately equal to the inflow rate through the channel 320 may eliminate suction that may otherwise be generated by channel 320. In some examples, the fluid 201 has an inflow rate of 130 mL/min through channel 320 and an outflow rate of 0 mL/min through channel 322. In other examples, an inflow rate may be 1-3 mL/sec through one of the channels, with little or no flow in the other channel. In other examples, the inflow rate through channel 320 may be approximately 150-200 mL/min and the outflow rate through channel 322 may be approximately 100-200 mL/min. Inflation may continue until the expandable device 300 engages with passageway 102 as shown in FIG. 3C. To carefully control the amount of time the expandable device is engaged with the tissue, inflation may occur rapidly. In some examples, inflation may occur over periods of approximately 1-10 seconds. In some examples inflation of expandable device 300 may take 2-5 seconds. Ablation may be a function of time and temperature, so when inflation occurs quickly, the time and temperature of ablation may be more easily controlled. In some examples, the flow rate into the expandable device may be five times greater during the inflation process 354 than the flow rate into the expandable device during the ablation contact process 356. In some examples a control system (e.g. control system 812) may directly monitor and control flow rates. In alternative examples, rather than controlling flow rates, the control system may instead regulate flow by monitoring and controlling pressures on the channels.

At process 356, the expandable device, in contact with the adjacent tissue along the anatomic passageway, ablates the tissue. Rapid inflation of the expandable device may allow the ablation start time to be precise and accurate providing for greater control over the length of treatment and desired ablation depth. In some examples, with reference to FIG. 3C, the expandable device 300, in a fully or substantially fully inflated state, maintains contact with the wall of the anatomic passageway 102 to ablate tissue with the inflated expandable device 300. While the expandable device 300 is in contact with the wall of the anatomic passageway 102, the inflow and outflow rates of fluid 201 may be approximately the same. Substantially equal inflow and outflow rates may allow the expandable device 300 to maintain its level of full or substantially full inflation. In some examples, there is a 30 mL/min inflow of fluid through the channel 320 and a 30 mL/min outflow through the channel 322. In other examples, there is a 40 mL/min inflow of fluid through the channel 320 and an outflow of 40 mL/min through the channel 322. Though the inflow and outflow rates can be the same or substantially the same, ablation can also occur with inflow and outflow rates that are slightly different. This can allow for slight inflation or deflation to occur during the process, expanding or retracting the contact with tissue being treated. This may be beneficial in treatments where target tissue at the inflation site requires more ablation than surrounding tissue. During the process 356, the temperature of the expandable device 300 may remain relatively constant due to the circulating fluid 201. While heated fluid cools during treatment as heat is transferred to the tissue, constant circulation of heated fluid 201 into the expandable device 300 and removal of cooled fluid 201 from the expandable device 300 may maintain the expandable device 300 at a constant temperature, allowing for precise control of ablation. Agitation or other turbulence in the fluid 201 may also help maintain temperature consistency through forced convection. Temperature consistency results in ablation consistency.

The expandable device 300 may be engaged with the tissue of the passageway 102 for a set amount of time at a set temperature. In some examples, the inflated expandable device 300 may remain in contact with passageway 102 for between approximately 1 and 30 seconds during ablation. In some examples, contact may have a duration of approximately 5 seconds while in other examples, contact may have a duration of approximately 20 seconds. In some examples, the temperature of the fluid 201 during process 356 may be between 50 and 85 degrees Celsius. The temperature of the fluid may remain constant throughout the process, thus causing the expandable device 300 to maintain a constant temperature. The above examples of ablation dosage (e.g., time and temperature) may be adjusted. In some examples, the ablation time and temperature settings can be adjusted based on different sizes of anatomic passages within a single anatomy to compensate for variables such as volume of the balloon.

In some examples, the temperature 304 of expandable device 300 may be varied throughout the duration of contact with the wall of the anatomic passageway 102. For example, the temperature may be decreased at a fixed rate so that the expandable device cools near the end of tissue contact period. If the temperature of the expandable device is lowered near the end of the tissue contact period, the risk of over ablation may be reduced. However, in other examples it may be useful for the temperature of expandable device 300 to increase near the end of the tissue contact period. In some examples, since inflation and deflation of expandable device 300 may occur rapidly, the temperature of expandable device 300 may stay constant throughout the tissue contact period. Rapid inflation and deflation of expandable device 300 may allow for precise control of contact time and thus ablation duration.

During the process 356, the sensing system 214 may monitor one or more parameters to ensure sufficient contact is made between expandable device 300 and anatomic passageway 102. One of the monitored parameters may be the inflation or pressure of expandable device 300. The sensing system 214 may also help facilitate adjustments in fluid flow. In one example, the sensing system 214 may measure fluid flow rate to monitor the fluid volume in expandable device 300 associated with sufficient contact with passageway 102. In some examples, the sensing system 214 may sense contact through use of a capacitive sensor, an imaging system, or a pressure sensor. The capacitive sensor may include capacitive touch sensors on a surface of the expandable device 300. An imaging system may provide image data that allows a user to visualize contact. The imaging system may include an endoscopic camera, an ultrasound, or fluoroscopy, among other examples. A pressure sensor may evaluate contact by sensing an increase or decrease in pressure. The pressure sensor may be placed on the inside of the expandable device 300 or may be a capacitive pressure sensor on the expandable device 300. In other examples the pressure sensor may be a contact switch on the expandable device 300 that opens or closes when the expandable device 300 reaches a certain inflation state. If the change in pressure is not attributable to the fluid inflow or outflow, it may be an indication that the expandable device 300 has made sufficient contact with passageway 102. In yet other examples artificial intelligence may be used to interpret video and/or image data of the expandable device 300 and anatomic passageway 102. The artificial intelligence may automatically analyze the video and/or image data to determine when the expandable device reaches a certain inflation state and/or makes contact with tissue.

During the process 356, the temperature gradient or variability across the surface of the expandable device 300 may be minimized. In some examples, a consistent temperature across the surface may be facilitated by increasing the circulation of fluid 201. In other examples, an agitation or duty cycle may be introduced to the flow of the fluid 201. For example, a duty cycle may be added to the flow rate through use of solenoid-based agitator of the agitator system 212. Alternatively, the fluid 201 within the expandable device 300 may be agitated or oscillated by vibrating the expandable device or activating an impellor within expandable device 300.

During the process 356, the amount of ablation that occurs can be adjusted for different sizes of anatomic passages within a single anatomy. To increase ablation, the time of contact between the expandable device and the wall of the anatomic passageway may be increased, the pressure applied to the wall of the anatomic passageway by the balloon may be increased, and/or the temperature of the expandable device may be increased. Any combination of time, temperature, and pressure may be used to achieve the desired ablation result. Optionally, one side of the elongated shaft 302 may be insulated to reduce ablation in one direction.

Ablation may cause a destruction of cells in tissue providing for the destroyed cells to naturally slough off and eventually allow for regrowth of healthy tissue. For some treatments, it may be advantageous to mechanically remove destroyed tissue cells rather than waiting for it to naturally slough off. Accordingly, referring to FIG. 3A, the expandable device 300 may have a rough surface texture to promote delamination between the epithelium and submucosa along the wall of the anatomic passage. In some examples, the expandable device 300 may be formed with the rough surface texture or may have the rough surface texture added after manufacturing. The rough surface may be attributed to bumps, ridges, and/or scales on the outer surface of expandable device 300. The rough surface may agitate and proactively remove goblet cells rather than waiting for natural slough off. To facilitate agitation, the elongated shaft 302 and expandable device 300 may be rotated.

At process 358, the expandable device may be deflated by decreasing the inflow rate relative to the outflow rate. As the expandable device deflates, it may disengage from the wall of the anatomic passage 102. For example and with reference to FIG. 3D, with the fluid inflow rate decreased relative to the fluid outflow rate, the expandable device 300 may disengage from the wall of anatomic passageway 102. The deflation and disengagement may occur rapidly to abruptly stop ablation. In some examples, deflation occurs in approximately 1-10 seconds, and in some examples, it may occur in 2 or 3 seconds. In some examples the outflow rate through channel 322 is 130 mL/min while the inflow rate through channel 320 is 0 mL/min. In other examples, channel 320 and channel 322 may both be used for outflow. For example, the outflow rate through channel 320 is 200 mL/min while the outflow rate through second channel 322 is 200 mL/min. In some examples, the flow rate out of the expandable device may be five times greater during the deflation process 358 than the flow rate out of the expandable device during the ablation contact process 356.

FIG. 3E illustrates the deflated expandable device 300. In the deflated state, the circulation of fluid 201 may cease. The expandable device 300 cools/partially cools prior to or as it is either removed from the passageway or moved to a different treatment location. The expandable device 300 may be re-heated as it is prepared to perform treatment on other target tissue. In other examples, expandable device 300 and the elongated shaft 302 may continue to be heated or partially heated as described above in process 352. In some examples, the expandable device may remain partially inflated. In yet other examples, a suction force may be applied to expandable device 300 to ensure that it remains in a fully or partially deflated state.

In some examples, the treatment device 220 may be moved (e.g., retracted) while the fluid 201 is circulating through the treatment device 220 (elongated shaft 302 and the expandable device 300). In some examples, the movement of the treatment device 220 may include coupling the treatment device 220 to an elongated delivery instrument and movement of the treatment device may be achieved by moving the elongated delivery instrument. In some embodiments, movement of the treatment device 220 and/or the elongated delivery instrument may be performed by manually manipulating a proximal portion of the treatment device/elongated delivery instrument. In some examples, the control system 216 (or control system 812 of the robot assisted medical system 800 described in FIG. 8) may move the treatment device 220 or elongated delivery instrument through the passageway 102 while the expandable device is in an inflated state so that ablation occurs along a length of the passageway. In some examples, the control system may move the treatment device while the expandable device is in a collapsed or under-inflated configuration. The expandable device may be re-inflated at a new location. This may allow ablation to be completed at different locations and throughout an entire airway or structure. In some examples, the elongated shaft 302 and/or the delivery device may be coupled to a manipulator of a robot-assisted medical system (e.g., a system 800) and movement of the treatment device from a first location to a second location may be performed by actuation of a manipulator.

If the circulating heated fluid raises the temperature on an outside surface of an outer wall of the elongated shaft for an extended period of time, the catheter may damage adjacent (e.g., healthy) anatomic tissue. Thus, an external temperature along the catheter may be reduced to minimize thermal risk to the adjacent tissue. At an optional process, expandable device 300 and/or elongated shaft 302 may be cooled after ablation has occurred. Cooling expandable device 300 and/or elongated shaft 302 may be achieved by circulating a cooled or ambient temperature fluid through expandable device 300. Cooling the elongated shaft 302 may prevent damage to the adjacent patient anatomy or to other components such as sensors, electronics, or imaging components in the catheter or in a sheath through which the catheter extends. In some examples, fluid heating system 208 may decrease the temperature of fluid 201 without adding or subtracting any fluid to the system. In other examples, the fluid heating system 208 may add fluid from a separate reservoir containing fluid that is cooler than the fluid circulated during the inflation and deflation process.

In some examples, an outside temperature of the outer wall of the catheter may be maintained at a pre-determined safety temperature of, for example, 70° C. by controlling the duration of the flow of the heated fluid and the duration of the flow of the cooled fluid to prevent outer wall temperatures from exceeding the safety temperature. Temperature sensors may be included within or along the outer wall of the catheter to measure temperature, and the duration of flow may be altered based on the sensed temperature, in a closed loop manner. In some examples, the flow rate, flow duration, and/or fluid temperature may be altered based on temperature of the catheter wall. The temperature of the fluid may be monitored (e.g., with a temperature sensor within the delivery fluid lumen) and may be used to adjust the temperature, flow rate, and/or duration of delivery of the circulating fluid. In some examples the temperature of the fluid may be monitored at various areas of the fluid system or catheter, such as at a proximal location, a distal location immediately before fluid entry/exit from the expandable device, or multiple points in between to determine change in temperature as fluid is delivered down the length of the catheter. Additionally, in some examples, the cooled fluid may circulate through a channel(s) of the catheter, after delivery of the heated treatment fluid, to maintain the temperature of the outer wall of the catheter at or below the safety temperature.

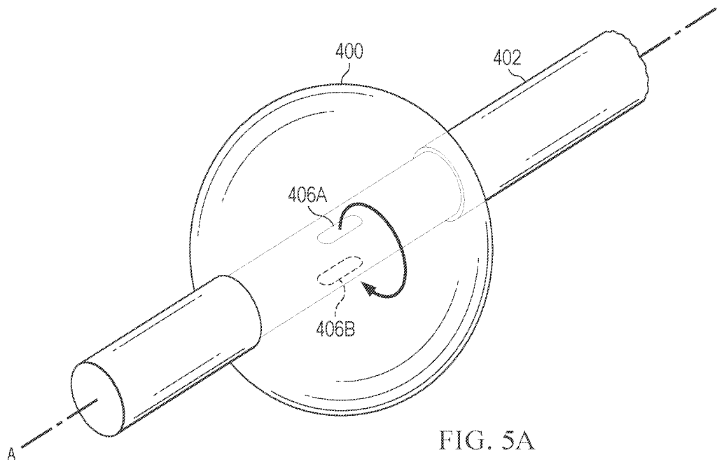

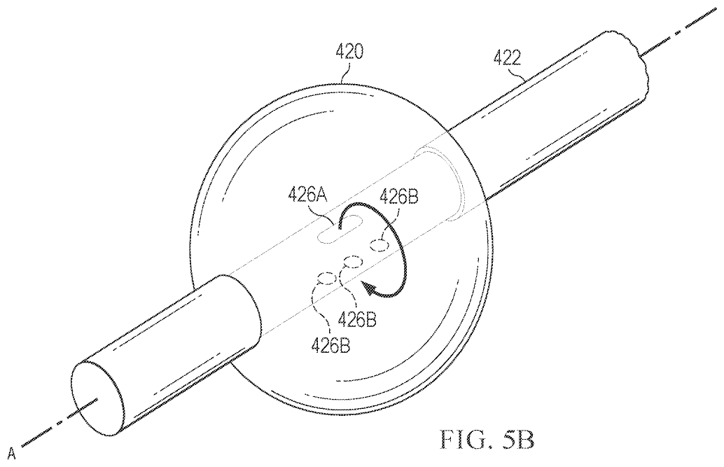

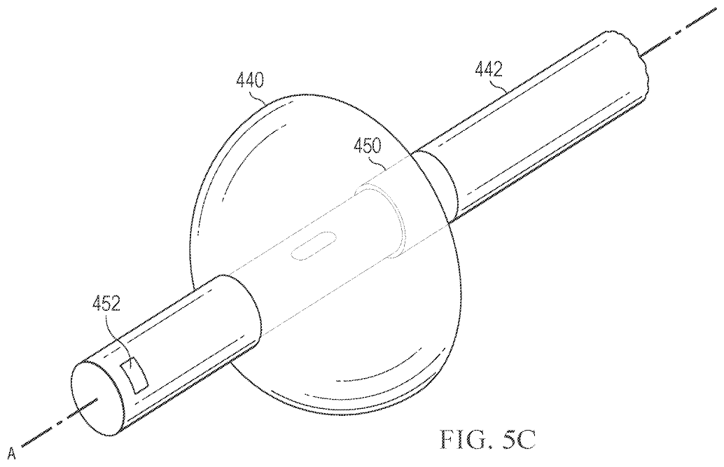

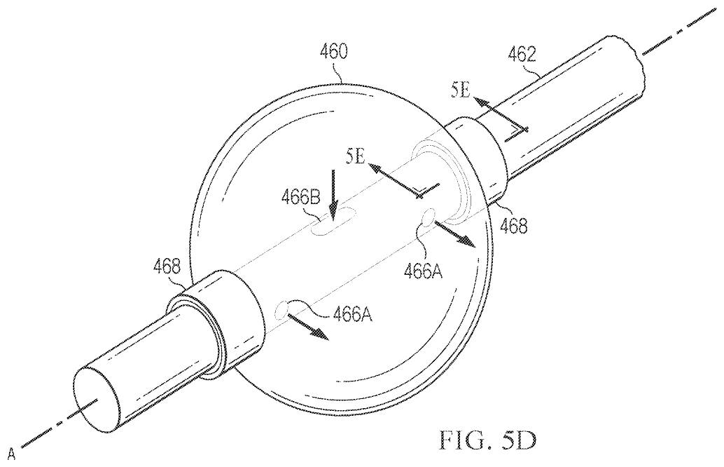

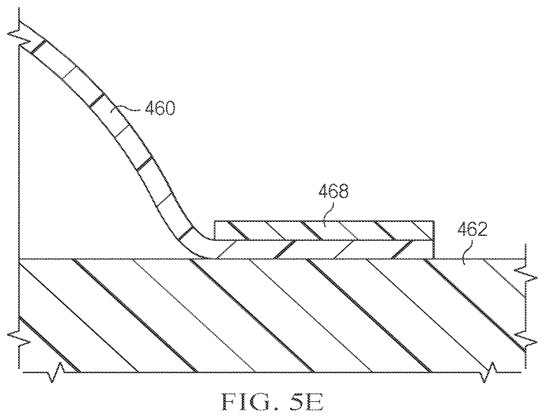

FIG. 5A illustrates a configuration of fluid ports, according to some examples. In this example, an elongated shaft 402 may be substantially similar to elongated shaft 302 except for the differences as explained. The elongated shaft 402 may carry an expandable device 400 which may be substantially similar to expandable device 300 except for the differences as explained. In this example, a fluid port 406A may be an inlet port that conveys fluid from the elongated shaft 402 into the expandable device 400. A fluid port 406B may be an outlet port that conveys fluid from the expandable device 400 into the elongated shaft 402. The fluid ports 406A and 406B may serve as either inlet or outlet ports. Flow may be reversed in the inlet and outlet ports so that an inlet port may convey fluid from the expandable device 400 into the elongated shaft 402 and an outlet port may convey fluid from the elongated shaft 402 into the expandable device 400. In some examples the fluid port 406A and 406B may simultaneously convey fluid into the elongated shaft 402 or into the expandable device 400. The fluid port 406A may be located approximately 180 degrees from fluid port 406B about the axis A of elongated shaft 402. In other examples, the inlet port may be located less than 180 degrees from the outlet port about the axis A. In yet other examples, inlet port may be located more than 180 degrees from the outlet port about the axis A.

FIG. 5B illustrates another configuration of the fluid ports, according to some examples. In this example, an elongated shaft 422 may be substantially similar to elongated shaft 302 except for the differences as explained. The elongated shaft 422 may carry an expandable device 420 which may be substantially similar to expandable device 300 except for the differences as explained. In this example, a fluid port 426A may be an inlet port that conveys fluid from the elongated shaft 422 into the expandable device 420. A plurality of fluid ports 426B may be outlet ports that convey fluid from the expandable device 420 into the elongated shaft 422. The fluid ports 426A and 426B may serve as either inlet or outlet ports. Flow may be reversed in the inlet and outlet ports so that an inlet port may convey fluid from the expandable device 420 into the elongated shaft 422 and an outlet port may convey fluid from the elongated shaft 422 into the expandable device 420. In some examples the fluid ports 426A and 426B may simultaneously convey fluid into the elongated shaft 422 or into the expandable device 420. In this example, three outlet ports are shown, but fewer or greater than three outlet ports may be suitable. In other examples, a plurality of fluid outlet ports may convey fluid into the expandable device, and a single fluid inlet port may return fluid to the elongated shaft. In other examples, a plurality of fluid outlet ports may convey fluid into the expandable device, and a plurality of fluid inlet ports may return fluid to the elongated shaft. In some examples, fluid inlet and or outlet ports may have any of a variety of different shapes and sizes. The shapes and sizes of the ports may be suitable for particular procedures. For example, port size and shape may vary based on the diameter of passageway 102, the type of treatment being performed, the diameter of the elongated shaft, the shape of the expandable device, the type of fluid, and the desired temperature of expandable device.

FIG. 5C illustrates an outer sheath 450 slidable along the exterior surface of an elongated shaft 442 to control the inflation shape of an expandable device 440. In this example, the elongated shaft 442 may be substantially similar to elongated shaft 302 except for the differences as explained. The elongated shaft 442 may carry an expandable device 440 which may be substantially similar to expandable device 300 except for the differences as explained. The outer sheath 450 may be longitudinally adjustable along the axis A to control the inflation shape of the expandable device 440. Sliding the outer sheath 450 toward the distal end of the elongated shaft 442 may reduce the unrestricted length of the expandable device, constraining inflation of expandable device 440. In other examples, the outer sheath 450 may be located distally of the expandable device and slid proximally to restrict the inflatable length and diameter of the expandable device. In other examples, multiple outer sheaths may be utilized to displace the expandable device 300 in multiple locations. The multiple outer sheaths 450 may displace the expandable device 300 simultaneously or could work independently at differing lengths along the axis A during the treatment. In yet other examples, outer sheath 450 may have openings or apertures. The openings may allow the expandable device to comply and take on various shapes, allowing for precise control of locations at which the expandable device would inflate. In some examples the outer sheath may be formed from a silicone tubing or polymer material. In some examples the outer sheath 450 may be the elongated delivery instrument (e.g., the robot-assisted instrument 804).

In some examples, a depth indicator 452 may be located at a distal portion of elongated shaft 442 to indicate how far a distal tip of the elongated shaft 442 is inserted relative to the delivery instrument. In other examples, the depth indicator may be located at a proximal portion of the elongated shaft 452. The depth indicator may be used with any of the catheter configurations described herein. A depth indicator may be visible by video using an imaging system or otherwise. In some examples, fluoroscopy may be used to indicate the depth relative to the delivery instrument. In some examples the depth indicator 452 may be a position sensor (e.g., EM sensor, fiber optic sensor) for determining position of the distal tip of the elongated shaft 442 in sensor space. In some examples the delivery instrument may have an EM sensor in the same magnetic field as the EM position sensor, making their relative positions known and obtainable. Where other types of position sensors are used, the sensor coordinate systems of the position sensors can be registered to determine relative insertion.

FIG. 5D illustrates another configuration of the fluid ports, according to some examples. In this example, an elongated shaft 462 may be substantially similar to elongated shaft 302 except for the differences as explained. The elongated shaft 462 may carry an expandable device 460 which may be substantially similar to expandable device 300 except for the differences as explained. In this example, fluid ports 466A may be spaced apart inlet ports positioned near proximal and distal ends of the expandable device 460. The fluid ports 466A convey fluid from the elongated shaft 462 into the expandable device 460. A fluid port 466B may be an outlet port that conveys fluid from the expandable device 460 into the elongated shaft 462. In this example, the spacing of the ports 466A, 466B may contribute to the agitation or chum of the fluid to provide a more uniform temperature across the surface of the expandable device 460.

FIG. 5D also illustrates a pair of cuffs 468 that may be used to secure the expandable device 460 to the elongated shaft 462. The cuffs 468 may be formed, for example, from a polyolefin or other heat shrink material. The cuffs 468 may fortify an adhesive coupling between the expandable device 460 and the shaft 462 and may extend the useful life of the assembly by preventing the expandable device 460 from peeling away from the shaft 462 over repeated cycles of inflation and deflation. As shown in the detailed cross-sectional view of FIG. 5E, the expandable device 460 may be sandwiched between the cuff 468 and the shaft 462. In some examples, a lamination material, such as a low density polyethylene or high density polyethylene material, may extend over the elongated shaft 462 and may extend into abutment with the cuffs 468. The cuffs and lamination may be used with any of the treatment devices described herein.

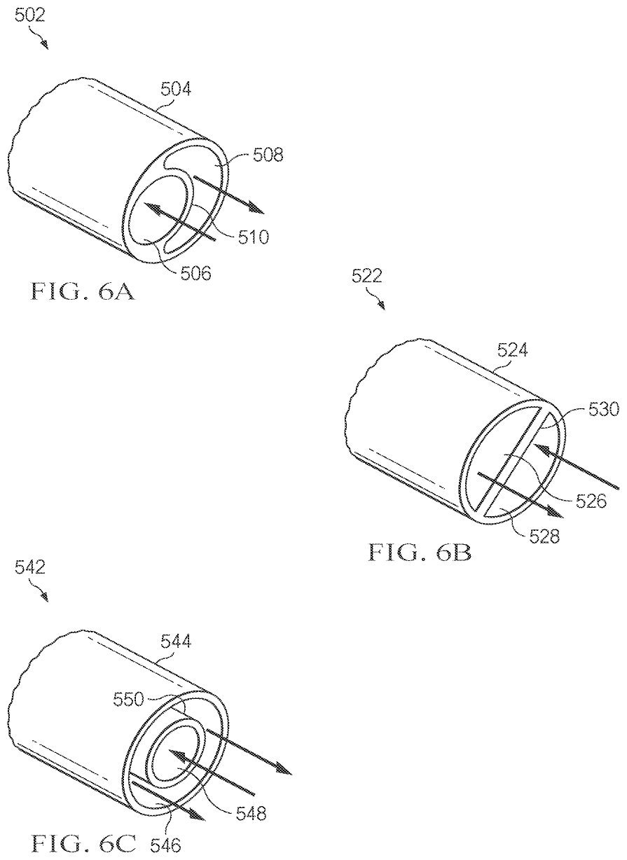

FIGS. 6A-6C illustrate various configurations of channels within an elongated shaft. Other configuration of channels may be suitable. In the example of FIG. 6A, an elongated shaft 502 may be substantially similar to elongated shaft 302 except for the differences as explained. The elongated shaft 502 includes an outer wall 504, a channel 506 which has a circular cross section, and a channel 508 which has a crescent-shaped cross section. A wall 510 bounds a portion of the channel 506 and separates the channel 506 from channel 508. In some examples, fluid (e.g., fluid 201) is conveyed from the fluid system (e.g., fluid system 202) through the channel 508 to an expandable device (e.g., expandable device 300) and is conveyed through the channel 506 from the expandable device to the fluid system. The direction of the fluid flow may be different for different procedures or different at different stages of an inflation cycle.

In the example of FIG. 6B, an elongated shaft 522 may be substantially similar to elongated shaft 302 except for the differences as explained. The elongated shaft 522 includes an outer wall 524, a channel 526 which has a semi-circular cross section, and a channel 528 which has a semi-circular cross section. A wall 530 separates the channel 526 from channel 528. In some examples, fluid (e.g., fluid 201) is conveyed from the fluid system (e.g., fluid system 202) through the channel 526 to an expandable device (e.g., expandable device 300) and is conveyed through the channel 528 from the expandable device to the fluid system. The direction of the fluid flow may be different for different procedures or different at different stages of an inflation cycle.

In the example of FIG. 6C, an elongated shaft 542 may be substantially similar to elongated shaft 302 except for the differences as explained. The elongated shaft 542 includes an outer wall 544, a channel 546 which has a circular cross section, and a channel 548 which has a hollow circle cross section. The channel 546 is co-axial with the channel 548 and extends within the channel 548. A wall 550 separates the channel 546 from channel 548. In some examples, fluid (e.g., fluid 201) is conveyed from the fluid system (e.g., fluid system 202) through the channel 546 to an expandable device (e.g., expandable device 300) and is conveyed through the channel 548 from the expandable device to the fluid system. The direction of the fluid flow may be different for different procedures or different at different stages of an inflation cycle.

In some examples, there may be more than two channels to facilitate flow of fluid to and from the expandable device. One or more of the channels, e.g. 506/508/526/528/546/548, may be formed by multiple smaller sub-channels (not shown) to increase the structural integrity of the elongated shaft, e.g. 502/522/542. The channels 506/508/526/528/546/548 may include one or more walls that act as dividers to separate portions of each respective channel. In other examples, additional channels may be used to facilitate sensors and other instruments. In other examples, the elongated shaft may be radiopaque or have radiopaque portions to help visualize the diameter of the expandable device and estimate its volume and contact with the anatomic passage 102.

The expandable device 300 may have features that promote targeted and consistent ablation. For example, the expandable device may be selected may have different wall thicknesses to control the inflation diameter at various locations of the expandable device. For example, the wall of expandable device 300 may be thicker at the proximal and distal ends to constrain the distal and proximal direction inflation. Other variations of wall thickness of expandable device 300 may be employed to produce different shapes of expandable device 300. In some examples, expandable device 300 may comprise nested balloons with an outer balloon containing heated fluid and an inner balloon containing other un-heated fluid. In some examples, the expandable device 300 may include a non-compliant inner member and a compliant outer member, the inner member being housed within the outer member. In some examples, expandable device 300 may include an impeller or other circulation system to circulate fluid 201.

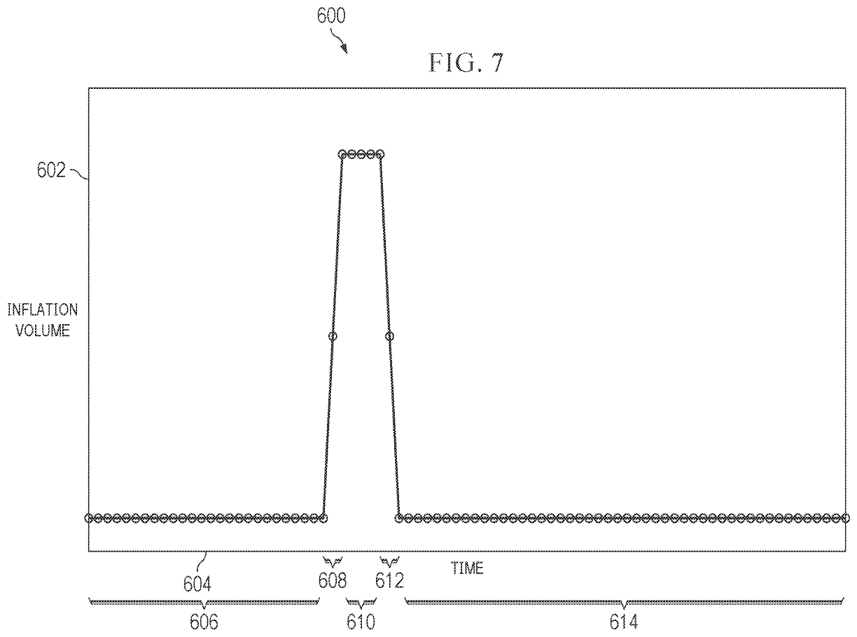

FIG. 7 is a graph 600 charting inflation volume 602 vs. time 604 throughout a minimally invasive thermal treatment via an instrument (e.g., treatment device 220) and expandable device (e.g., expandable device 300). Period 606 may be a heating stage, as described at process 352, in which the catheter and expandable device are heated by a circulating heated fluid. In this stage, the expandable device may not be inflated or in contact with the wall of the anatomic passageway.

Period 608 may be an inflation stage, as described at process 354, in which the expandable device is inflated with the heated fluid. A rapid increase in inflation volume may occur during this period, which may be relatively short (e.g., 2-3 seconds).

Period 610 may be a contact stage, as described at process 356, in which the expandable device is in contact with the anatomic passageway to ablate the localized area of the passageway. The inflation volume may be relatively constant during this period. The duration of this period may be procedure-dependent based upon the patient anatomy, disease characteristics, or other treatment factors.

Period 612 may be a deflation stage, as described at process 358, in which the expandable device is deflated by withdrawing some or all of the fluid from the expandable device. A rapid decrease in inflation volume may occur during this period, which may be relatively short (e.g., 2-3 seconds).

Period 614 may be a post-ablation stage in which the inflated device is deflated, having little or no inflation volume. The catheter and expandable device may be removed during the period 614 or navigated to a new location in the anatomic passageway.

In some examples, the systems and methods disclosed herein may be used in a medical procedure performed with a robot-assisted medical system as described in further detail below. As shown in FIG. 8, a robot-assisted medical system 800 may include a manipulator assembly 802 for operating a medical instrument 804 (e.g., medical instrument system 100, elongated delivery instrument, treatment device 220, or any of the instruments described herein) in performing various procedures on a patient P positioned on a table T in a surgical environment 801. The manipulator assembly 802 may be teleoperated, non-teleoperated, or a hybrid teleoperated and non-teleoperated assembly with select degrees of freedom of motion that may be motorized and/or teleoperated and select degrees of freedom of motion that may be non-motorized and/or non-teleoperated. A master assembly 806, which may be inside or outside of the surgical environment 801, generally includes one or more control devices for controlling manipulator assembly 802. Manipulator assembly 802 supports medical instrument 804 and may optionally include a plurality of actuators or motors that drive inputs on medical instrument 804 in response to commands from a control system 812. The actuators may optionally include drive systems that when coupled to medical instrument 804 may advance medical instrument 804 into a naturally or surgically created anatomic orifice. Other drive systems may move the distal end of medical instrument in multiple degrees of freedom, which may include three degrees of linear motion (e.g., linear motion along the X, Y, Z Cartesian axes) and in three degrees of rotational motion (e.g., rotation about the X, Y, Z Cartesian axes). The manipulator assembly 802 may support various other systems for irrigation, treatment, or other purposes. Such systems may include fluid systems (including, for example, reservoirs, heating/cooling elements, pumps, and valves), generators, lasers, interrogators, and ablation components.

Robot-assisted medical system 800 also includes a display system 810 for displaying an image or representation of the surgical site and medical instrument 804 generated by a sensor system 808 which may include an endoscopic imaging system. Display system 810 and master assembly 806 may be oriented so an operator O can control medical instrument 804 and master assembly 806 with the perception of telepresence. Any of the previously described graphical user interfaces may be displayable on a display system 810 and/or a display system of an independent planning workstation.

The sensor system 808 may include a position/location sensor system (e.g., an actuator encoder or an electromagnetic (EM) sensor system) and/or a shape sensor system (e.g., an optical fiber shape sensor) for determining the position, orientation, speed, velocity, pose, and/or shape of the medical instrument 804. The sensor system 808 may also include temperature, pressure, force, or contact sensors or the like. In some examples, the sensors systems 214 and 808 may be integrated or cooperative.

Robot-assisted medical system 800 may also include control system 812. Control system 812 includes at least one memory 816 and at least one computer processor 814 for effecting control between medical instrument 804, master assembly 806, sensor system 808, and display system 810. Control system 812 also includes programmed instructions (e.g., a non-transitory machine-readable medium storing the instructions) to implement a plurality of operating modes of the robot-assisted medical system including a navigation planning mode, a navigation mode, and/or a procedure mode. Control system 812 also includes programmed instructions (e.g., a non-transitory machine-readable medium storing the instructions) to implement some or all of the processes described in accordance with aspects disclosed herein, including, for example, expanding the expandable device, regulating the temperature of the heating system, regulating valves to control fluid delivery, controlling fluid flow rate, controlling insertion and retraction of the treatment instrument, controlling actuation of a distal end of the treatment instrument, receiving sensor information, altering signals based on the sensor information, selecting a treatment location, and/or determining a size to which the expandable device may be expanded.

Control system 812 may optionally further include a virtual visualization system to provide navigation assistance to operator O when controlling medical instrument 804 during an image-guided surgical procedure. Virtual navigation using the virtual visualization system may be based upon reference to an acquired pre-operative or intra-operative dataset of anatomic passageways. The virtual visualization system processes images of the surgical site imaged using imaging technology such as computerized tomography (CT), magnetic resonance imaging (MRI), fluoroscopy, thermography, ultrasound, optical coherence tomography (OCT), thermal imaging, impedance imaging, laser imaging, nanotube X-ray imaging, and/or the like. The control system 812 may use a pre-operative image to locate the target tissue (using vision imaging techniques and/or by receiving user input) and create a pre-operative plan, including an optimal first location for performing bronchial passageway and vasculature occlusion. The pre-operative plan may include, for example, a planned size to expand the expandable device, a treatment duration, a treatment temperature, and/or multiple deployment locations.

In the description, specific details have been set forth describing some examples. Numerous specific details are set forth to provide a thorough understanding of the examples. It will be apparent, however, to one skilled in the art that some examples may be practiced without some or all these specific details. The specific examples disclosed herein are meant to be illustrative but not limiting. One skilled in the art may realize other elements that, although not specifically described here, are within the scope and the spirit of this disclosure.

Elements described in detail with reference to one example, implementation, or application optionally may be included, whenever practical, in other examples, implementations, or applications in which they are not specifically shown or described. For example, if an element is described in detail with reference to one example and is not described with reference to a second example, the element may nevertheless be claimed as included in the second example. Thus, to avoid unnecessary repetition in the following description, one or more elements shown and described in association with one example, implementation, or application may be incorporated into other examples, implementations, or aspects unless specifically described otherwise, unless the one or more elements would make an example or implementation non-functional, or unless two or more of the elements provide conflicting functions. Not all the illustrated processes may be performed in all examples of the disclosed methods. Additionally, one or more processes that are not expressly illustrated in may be included before, after, in between, or as part of the illustrated processes. In some examples, one or more of the processes may be performed by a control system or may be implemented, at least in part, in the form of executable code stored on non-transitory, tangible, machine-readable media that when run by one or more processors may cause the one or more processors to perform one or more of the processes.

Any alterations and further modifications to the described devices, instruments, methods, and any further application of the principles of the present disclosure are fully contemplated as would normally occur to one skilled in the art to which the disclosure relates. In addition, dimensions provided herein are for specific examples and it is contemplated that different sizes, dimensions, and/or ratios may be utilized to implement the concepts of the present disclosure. To avoid needless descriptive repetition, one or more components or actions described in accordance with one illustrative example can be used or omitted as applicable from other illustrative examples. For the sake of brevity, the numerous iterations of these combinations will not be described separately. For simplicity, in some instances the same reference numbers are used throughout the drawings to refer to the same or like parts.

The systems and methods described herein may be suited for imaging, via natural or surgically created connected passageways, in any of a variety of anatomic systems, including the lung, colon, the intestines, the stomach, the liver, the kidneys and kidney calices, the brain, the heart, the circulatory system including vasculature, and/or the like. While some examples are provided herein with respect to medical procedures, any reference to medical or surgical instruments and medical or surgical methods is non-limiting. For example, the instruments, systems, and methods described herein may be used for non-medical purposes including industrial uses, general robotic uses, and sensing or manipulating non-tissue work pieces. Other example applications involve cosmetic improvements, imaging of human or animal anatomy, gathering data from human or animal anatomy, and training medical or non-medical personnel. Additional example applications include use for procedures on tissue removed from human or animal anatomies (without return to a human or animal anatomy) and performing procedures on human or animal cadavers. Further, these techniques can also be used for surgical and nonsurgical medical treatment or diagnosis procedures.

One or more elements in examples of this disclosure may be implemented in software to execute on a processor of a computer system such as control processing system. When implemented in software, the elements of the examples of this disclosure may be code segments to perform various tasks. The program or code segments can be stored in a processor readable storage medium or device that may have been downloaded by way of a computer data signal embodied in a carrier wave over a transmission medium or a communication link. The processor readable storage device may include any medium that can store information including an optical medium, semiconductor medium, and/or magnetic medium. Processor readable storage device examples include an electronic circuit; a semiconductor device, a semiconductor memory device, a read only memory (ROM), a flash memory, an erasable programmable read only memory (EPROM); a floppy diskette, a CD-ROM, an optical disk, a hard disk, or other storage device. The code segments may be downloaded via computer networks such as the Internet, Intranet, etc. Any of a wide variety of centralized or distributed data processing architectures may be employed. Programmed instructions may be implemented as a number of separate programs or subroutines, or they may be integrated into a number of other aspects of the systems described herein. In some examples, the control system may support wireless communication protocols such as Bluetooth, Infrared Data Association (IrDA), HomeRF, IEEE 802.11, Digital Enhanced Cordless Telecommunications (DECT), ultra-wideband (UWB), ZigBee, and Wireless Telemetry.

Note that the processes and displays presented might not inherently be related to any particular computer or other apparatus. Various general-purpose systems may be used with programs in accordance with the teachings herein, or it may prove convenient to construct a more specialized apparatus to perform the operations described. The required structure for a variety of these systems will appear as elements in the claims. In addition, the examples of the invention are not described with reference to any particular programming language. It will be appreciated that a variety of programming languages may be used to implement the teachings of the invention as described herein.

This disclosure describes various instruments, portions of instruments, and anatomic structures in terms of their state in three-dimensional space. As used herein, the term position refers to the location of an object or a portion of an object in a three-dimensional space (e.g., three degrees of translational freedom along Cartesian x-, y-, and z-coordinates). As used herein, the term orientation refers to the rotational placement of an object or a portion of an object (e.g., in one or more degrees of rotational freedom such as roll, pitch, and/or yaw). As used herein, the term pose refers to the position of an object or a portion of an object in at least one degree of translational freedom and to the orientation of that object or portion of the object in at least one degree of rotational freedom (e.g., up to six total degrees of freedom). As used herein, the term shape refers to a set of poses, positions, or orientations measured along an object.

While certain illustrative examples of the invention have been described and shown in the accompanying drawings, it is to be understood that such examples are merely illustrative of and not restrictive on the broad invention, and that the examples of the invention are not limited to the specific constructions and arrangements shown and described, since various other modifications may occur to those ordinarily skilled in the art.

1. A system comprising:
an elongate instrument including a first channel and a second channel;
an expandable device coupled to a distal portion of the elongate instrument, the expandable device in fluid communication with the first and second channels;
a pump system for circulating a fluid through the first channel and second channel;
a heating system for heating the fluid;
a drainage system coupled to the elongate instrument for removing the fluid from the expandable device, the drainage system configured to restrict a drainage flow; and
a control system coupled to the heating system and the pump system, the control system configured to control temperature and delivery of the heated fluid through the first channel and the second channel to:
heat the elongate instrument and the expandable device with the heated fluid while maintaining the expandable device in an uninflated configuration;
inflate the expandable device with the heated fluid;
ablate an anatomic passage with the inflated and heated expandable device; and
deflate the expandable device.

2. The system of claim 1, further comprising a sensor system coupled to the control system,
wherein the sensor system includes a volumetric flow sensor configured to sense a flow volume in at least one of the first channel and second channel, and
wherein the delivery of the heated fluid includes controlling a first flow rate through the first channel and a second flow rate through the second channel based on the flow volume.

3. The system of claim 1, further comprising a sensor system coupled to the control system,
wherein the sensor system includes a first sensor to sense a first pressure in the first channel and a second sensor to sense a second pressure in the second channel, and
wherein the delivery of the heated fluid includes controlling a first flow rate through the first channel and a second flow rate through the second channel based on the first pressure and the second pressure.

4. The system of claim 2, wherein the distal portion of the elongate instrument comprises a fluid inlet configured to pass the heated fluid from the first channel into the expandable device at the first flow rate and comprises a fluid outlet configured to pass the heated fluid from the expandable device into the second channel at the second flow rate.

5. The system of claim 4, wherein heating the elongate instrument and the expandable device includes controlling the first flow rate and the second flow rate.

6. The system of claim 5, wherein the first flow rate is substantially the same as or greater than the second flow rate.

7. The system of claim 4, wherein the first flow rate is greater than the second flow rate when inflating the expandable device.

8. The system of claim 2, wherein ablating includes maintaining an inflation state of the expandable device by controlling the first flow rate and the second flow rate.

9. The system of claim 8, wherein maintaining the inflation state includes delivering the heated fluid at the first flow rate through the first channel and evacuating the heated fluid at the second flow rate through the second channel, wherein the second flow rate is less than the first flow rate.

10. The system of claim 2, wherein inflating the expandable device includes delivering the heated fluid at the first flow rate through the first channel and delivering the heated fluid at the second flow rate through the second channel, wherein the first flow rate is approximately the same as the second flow rate.

11. The system of claim 1, wherein inflating includes delivering the heated fluid at a first rate through the first channel and ablating includes delivering the heated fluid at a second rate through the first channel, wherein the first rate is approximately five times greater than the second rate.

12. The system of claim 1, wherein deflating includes evacuating the heated fluid from the first channel and the second channel at substantially the same rate.

13. The system of claim 1, further comprising an agitator system to agitate the heated fluid delivered through the first channel or the second channel, wherein the agitator system is coupled to the control system and the elongate instrument.

14-15. (canceled)

16. The system of claim 1, wherein the first channel defines a plurality of first sub-channels or the second channel defines a plurality of second sub-channels.

17. (canceled)

18. The system of claim 1, wherein the expandable device includes a non-compliant inner member and a compliant outer member, the inner member being housed within the outer member.

19. (canceled)

20. The system of claim 1, further comprising a sensor system configured to monitor the inflation of the expandable device or a temperature of the heated fluid.

21-24. (canceled)

25. The system of claim 1, wherein the pump system includes a rotary pump or a syringe pump.

26. The system of claim 1, wherein the drainage system restricts the drainage flow by applying a back pressure to fluid exiting the elongate instrument.

27. The system of claim 1, wherein the drainage system includes a flow restriction device configured to control a size of the expandable device or a stage of a tissue treatment.

28. The system of claim 27, wherein the flow restriction device includes an orifice sized based on a size or volume of the expandable device.

29-42. (canceled)
